(19) United States
(12) Patent Application Publication
(10) Pub. No.: US 2025/0281723 A1
(43) Pub. Date: Sep. 11, 2025

(54) Percutaneous Catheter Fixation Ladder Lock

(71) Applicant: Abiomed, Inc., Danvers, MA (US)

(72) Inventor: Christopher Sheils, Danvers, MA (US)

(73) Assignee: Abiomed, Inc., Danvers, MA (US)

(21) Appl. No.: 19/051,352

(22) Filed: Feb. 12, 2025

Related U.S. Application Data
(60) Provisional application No. 63/561,533, filed on Mar. 5, 2024.

(51) Int. Cl. A61M 25/02; A61M 60/13; A61M 60/90

(52) U.S. Cl. CPC A61M 25/02; A61M 2025/028; A61M 2025/0286; A61M 60/13; A61M 60/90; A61M 2205/0216; A61M 2209/088; A61M 2210/04; A61M 2210/125

(57) ABSTRACT

A stabilization device for securing a catheter within cardiovascular system of a patient. The stabilization device includes a base defining a skin contacting surface, and a ladder-lock coupled to the base. The ladder-lock includes a pair of rails and one or more rungs extending between the rails, each of the rails being pivotably attached to the base such that the ladder-lock is pivotable from an unlocked position in which the catheter is slidable within the cardiovascular system of the patient and a locked position in which the catheter is fixed relative to the cardiovascular system.

### CROSS-REFERENCE TO RELATED APPLICATION

This application claims the benefit of the filing date of U.S. Provisional Patent Application No. 63/561,533, filed on Mar. 5, 2024, the disclosure of which is hereby incorporated herein by reference.

### BACKGROUND OF THE DISCLOSURE

The present disclosure relates to catheter fixation devices, and more particularly, to a fixation device that allows a physician to efficiently stabilize a repositioning catheter within the cardiovascular system of a patient.

Blood pump assemblies, such as intracardiac or intravascular blood pumps, may be introduced in the heart to deliver blood from the heart into an artery. Such mechanical circulatory support devices are often introduced to support the function of the heart after a patient suffers a cardiac episode. One such class of devices is the set of devices known as the IMPELLA® family of devices designed by Abiomed, Inc. of Danvers, MA. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY OF THE DISCLOSURE

In accordance with a first aspect of the present disclosure, a catheter fixation apparatus including a ladder-lock is provided. Among other advantages, the ladder-lock may be quickly and efficiently transitioned between an unlocked position, in which a physician can reposition an intracardiac pump, and a locked position in which the intracardiac pump is stabilized against movement within the cardiovascular system of the patient.

One embodiment of the catheter fixation apparatus includes a base defining a skin contacting surface; and a ladder-lock coupled to the base. The ladder-lock is formed of a pair of rails and one or more rungs extending between the rails, each of the rails being pivotably attached to the base such that the ladder-lock is pivotable from an unlocked position in which the catheter is slidable within a cardiovascular system of a patient, and a locked position in which the catheter is fixed relative to the cardiovascular system.

The base may be an adhesive pad, and the skin contacting surface of the adhesive pad may include a medical grade adhesive configured to be adhered to skin of the patient. Alternatively, the base may be formed of a material configured to be sutured to skin of the patient such as a fabric or a medical grade plastic defining one or more eyelets.

The one or more rungs may include a plurality of rungs and the plurality of rungs may be spaced apart from one another such that the catheter is configured to extend over at least a first one of the rungs and underneath at least a second one of the rungs. In some instances, the fixation device may include a spring biasedly arranged against one of the rails to stabilize the ladder-lock against unintended movement.

The fixation device may further include at least one fastener to secure the ladder-lock in the locked position. The fastener may be substantially U-shaped and formed of a resilient material arranged to receive the one or more rungs to mechanically secure the ladder-lock in the locked position. Alternatively, the fixation device may further include at least one latch movably arranged to cover at least one of the rails, or the one or more rungs, to secure the ladder-lock in the locked position.

A sheath assembly is also provided herein and includes a catheter including a sheath for insertion into a cardiovascular system of a patient, the sheath being configured to receive at least a portion of an intravenous medical device; and a fixation apparatus for stabilizing the catheter within the cardiovascular system of the patient. The fixation apparatus includes a base defining a skin contacting surface; and a ladder-lock coupled to the base, the ladder-lock including a pair of rails and one or more rungs extending between the rails. Each of the rails are attached to the base and movable between a first position in which the catheter is slidable within the cardiovascular system and a second position in which the catheter is compressed by the ladder-lock and fixed relative to the cardiovascular system.

In some instances, the catheter may be a repositioning catheter and/or the intravenous medical device may be an intracardiac pump. Additionally, the base may be an adhesive pad and/or the base may be formed of a material configured to be sutured to skin of the patient. The one or more rungs may include a plurality of rungs and be spaced apart from one another such that the catheter is configured to be weaved over at least a first one of the rungs and underneath at least a second one of the rungs.

A method of delivering and stabilizing a medical device within a cardiovascular system of a patient is also provided herein and includes: delivering a catheter to a target site through the cardiovascular system of the patient, the catheter holding the medical device; and transitioning a ladder-lock attached to a base from an unlocked position in which the catheter is slidable within the cardiovascular system of the patient to a locked position in which a portion of the catheter is secured by the ladder-lock to fix the catheter relative to the cardiovascular system of the patient.

The method may include determining that the intracardiac pump is mispositioned, transitioning the ladder-lock from the locked position to the unlocked position, sliding the catheter to reposition the intracardiac pump within a native valve, and transitioning the ladder-lock from the unlocked position to the locked position.

The method may further include securing the base to skin of a patient via an adhesive or a suture. Additionally, the method may include weaving the catheter over a first rung of the ladder-lock and underneath a second rung of the ladder-lock. In some instances, the medical device may be an intracardiac pump and/or the target site may be across a native valve.

### BRIEF DESCRIPTION OF THE DRAWINGS

Various embodiments of the present disclosure are described herein with reference to the drawings, wherein:

FIG. 1 is a highly schematic cutaway view of the human heart, showing a transfemoral approach for delivering an intracardiac heart pump to the aortic valve;

FIG. 2 is perspective view of an intracardiac heart pump, an introducer sheath assembly, and a repositioning sheath assembly, in accordance with an embodiment of the present disclosure;

FIG. 3 is a highly schematic cutaway view of the human showing the intracardiac heart pump of FIG. 2 properly positioned within the native aortic valve;

FIGS. 4A-4C are perspective views illustrating a ladder-lock securement device in accordance with an embodiment of the present disclosure;

FIG. 5 is an elevation view of a ladder-lock securement device in accordance with another embodiment of the present disclosure; and

FIG. 6 is an elevation view of a ladder-lock securement device in accordance with yet another embodiment of the present disclosure.

### DETAILED DESCRIPTION

Intracardiac pump assemblies can be introduced into the heart either surgically or percutaneously and used to pump blood from one location in the heart or circulatory system to another location in the heart or circulatory system. When deployed in the heart, for example, an intracardiac pump can transfer blood from the left ventricle to the aorta, or from the inferior vena cava to the pulmonary artery. Traditionally, intracardiac pumps operate in parallel with the native heart to supplement cardiac output and partially or fully unload components of the heart. For this reason, intracardiac pumps are often utilized in instances of cariogenic shock, during high-risk percutaneous coronary intervention (PCI), right heart failure, congestive heart failure, or severe lung failure, to relieve stress on the heart during recovery or while the patient awaits a heart transplant. Examples of such systems include the IMPELLA® family of devices designed by Abiomed, Inc., Danvers Mass.

Several IMPELLA® devices are of relatively small circumferential size and can be percutaneously delivered into a patient less invasively than intracardiac pumps that are introduced surgically. When delivered percutaneously, these intracardiac pumps may be inserted into a patient and delivered via a tube-like delivery device such as a repositioning catheter.

During delivery, the repositioning catheter is tracked through the vasculature of a patient to advance the intracardiac pump to a target site (e.g., across the aortic valve in the case of “left-side” intracardiac devices, or across the pulmonary valve in the case of “right-side” intracardiac devices). Echocardiography and/or other traditional imaging techniques may be utilized during delivery to properly position the intracardiac pump. Once in position, the intracardiac pump may be turned on to suction blood through an inflow portion (e.g., an inflow cage) of the intracardiac device, and to expel the pumped blood from the outflow portion (e.g., an outflow cage) of the intracardiac device. In the case of left-side intracardiac pumps, the inflow cage of the intracardiac device may be positioned within the left ventricle of the heart, and the outflow cage of the intracardiac pump may be positioned within the aorta. On the other hand, in the case of right-side intracardiac pumps, the inflow cage of the intracardiac device may be positioned within the inferior vena cava, and the outflow cage of the intracardiac device may be positioned within the pulmonary artery.

The clinical success of intracardiac pumps is dependent, in part, on proper positioning of the pump. For example, in the case of left-side intracardiac pumps, if the inflow cage is not properly positioned within the left ventricle, and without obstruction from anatomical structures such as the ventricular wall or the mitral valve, the intracardiac pump will inefficiently suction blood from the left ventricle. Additionally, if the outflow cage of the intracardiac pump is not sufficiently positioned within the aorta, the pumped blood may be susceptible to returning to the left ventricle, causing further stress on the heart. As a result, it is important for percutaneous intracardiac pumps to be stabilized in the proper position over the course of the entire treatment. For this reason, repositioning catheters often include a butterfly structure that can be secured to the skin of a patient and a securement device, such as a conventional Tuohy-Borst type device, to prevent the catheter from moving after the intracardiac pump has been properly positioned.

Despite the improvements that have been made to percutaneous intracardiac pump and repositioning sheath assemblies, shortcomings remain. For example, slack may be formed in the catheter as the intracardiac pump is tracked around curves of the vasculature on its way to the target site within the heart. The inventors have recognized, that even when a proximal end of the sheath is sutured or otherwise secured to the skin of the patient, ambulation or other movement of the patient can alter the amount of slack in the catheter, in turn, causing the intracardiac pump to migrate from a desired position (i.e., become mispositioned), and result in inefficiencies of the intracardiac pump. In some instances, patient movement can even damage the sutures, resulting in further migration of the catheter. Accordingly, the inventors have recognized and appreciated numerous benefits associated with the external catheter fixation devices disclosed herein.

When deployed in the heart, an intracardiac device collects blood from one area of the heart and pumps the blood to another area of the heart, to assist the heart in performing its normal function. As used herein in connection with an intracardiac pump, the term “inflow” refers to the portion of the intracardiac pump through which blood enters the pump, and the term “outflow” refers to the portion of the intracardiac pump through which blood is expelled. When used in connection with devices for delivering the intracardiac pump into a patient, the terms “proximal” and “distal” are to be taken as relative to the user of the delivery devices. For example, “proximal” or “proximal end” is to be understood as relatively close to the operator, and “distal” or “distal end” is to be understood as relatively farther away from the operator. Also as used herein, the terms “substantially,” “generally,” “approximately,” and “about” are intended to mean that slight deviations from absolute are included within the scope of the term so modified.

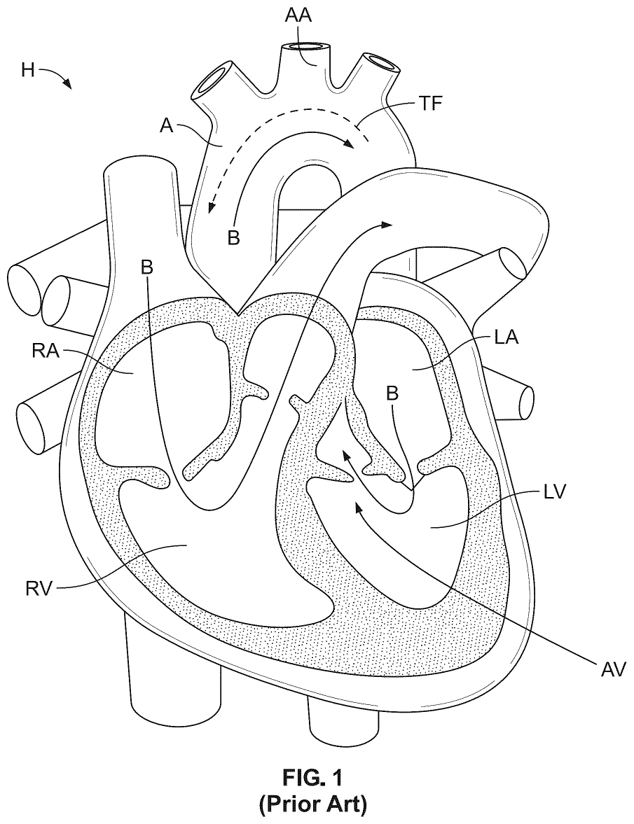

FIG. 1 is a schematic cutaway representation of a human heart H. The human heart includes two atria and two ventricles: right atrium RA and left atrium LA, and right ventricle RV and left ventricle LV. Heart H further includes aorta A and an aortic arch AA. Disposed between left ventricle LV and aorta A is aortic valve AV. The aortic valve, also known as the left semilunar valve or the left arterial valve, generally includes three leaflets that coapt to regulate blood flow between left ventricle LV and aorta A. When left ventricle LV contracts during systole, aortic valve AV opens, and blood is pushed from the left ventricle through aorta A to major arteries of the vasculature system. Blood flows through heart H in the direction shown by arrows “B”.

A dashed arrow, labeled “TF”, indicates a transfemoral approach of delivering an intracardiac pump to a target site, in this case to a location across aortic valve AV. Using a transfemoral approach, the intracardiac pump is inserted into the femoral artery, tracked through the vasculature, and then introduced to the target site via aortic arch AA. Echocardiography and other means may be used to help guide a delivery device, such as a repositioning catheter, to the target site. Other approaches, such as an axially approach (i.e., via the axillary artery), are possible for delivering the intracardiac pump to aortic valve AV, or to other target sites within heart H.

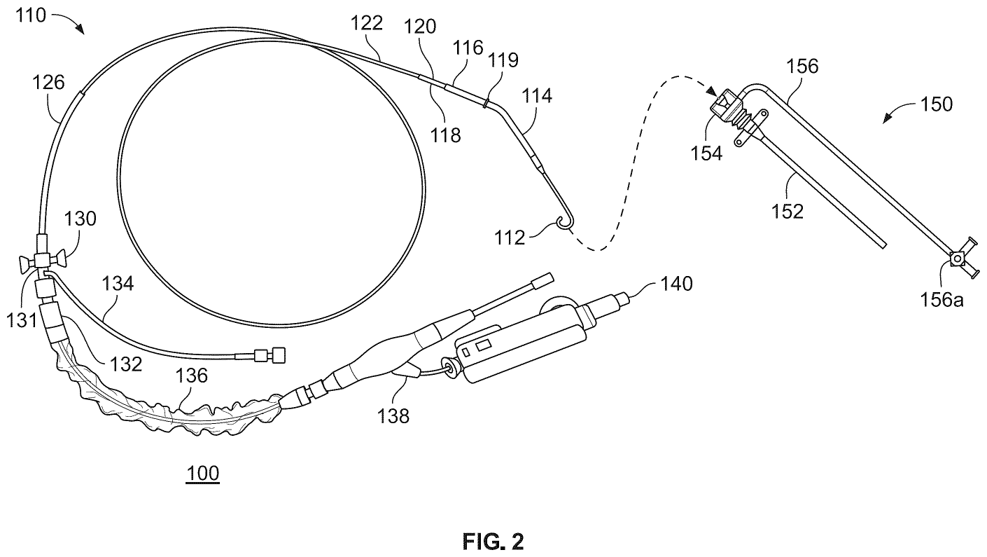

FIG. 2 illustrates a system 100 including an intracardiac pump and repositioning sheath assembly 110, and an introducer sheath assembly 150. A handle 138 may be provided at the proximal end of intracardiac pump and repositioning sheath assembly 110. Handle 138 may be operably coupled to the proximal end of catheter 122 and, therefore, arranged to advance the catheter within the cardiovascular system of a patient and to retract the catheter from the cardiovascular system of the patient, unless movement of the catheter is fixed by a securement device 132 as will be explained in further detail hereinafter.

Catheter 122 may be enclosed by a protective sleeve 136 extending between handle 138 and securement device 132. Put differently, the proximal end of protective sleeve 136 may be attached to handle 138, and the distal end of the protective sleeve may be attached to securement device 132, using any technique known in the art, to enclose and protect a proximal portion of catheter 122. Protective sleeve 136 may be formed of any material, such as a medical grade plastic, suitable for preventing the proximal end of catheter 122 from being contaminated as the catheter is advanced into the vasculature of a patient.

A hemostasis valve 131 may be provided between a distal end of securement device 132, and a proximal end of butterfly 130. Hemostasis valve 131, securement device 132, and butterfly 130 may be removably coupled from one another or manufactured as a single unitary component. A proximal end of repositioning sheath 126 is coupled to butterfly 130. Repositioning sheath 126 defines a lumen extending from the proximal end of the repositioning sheath to a distal end of the repositioning sheath that is sized to slidably receive catheter 122 therethrough.

Intracardiac pump and repositioning sheath assembly 110 may also include a purge fluid port 140. The purge fluid port 140 is in communication with handle 138 and is arranged to provide purge fluid (e.g., a solution of dextrose or glucose with heparin) to a purge lumen (not shown) within catheter 122. The purge lumen of catheter 122 is in fluid communication with, and configured to deliver the purge fluid to, the motor housing 120 of the intracardiac pump. Motor housing 120 may include a motor and an impeller. In some embodiments, the motor may be external to the patient, in which case catheter 122 may enclose a flexible drive, such as a shaft or cable, and motor housing 120 may enclose an impeller connected to that drive shaft or cable.

With continued reference to FIG. 2, the distal end of motor housing 120 may be coupled to a blood outflow cage 118. The distal end of blood outflow cage 118 may be coupled to a cannula 116, which in turn is coupled a proximal end of blood inflow cage 114. Cannula 116 may include a marking 119, such as a radiopaque marker that is visible under fluoroscopy, to assist a clinician in properly positioning the cannula 116 within the native heart valve as shown in FIG. 3. In some embodiments, cannula 116 may be expandable. An atraumatic extension, such as pigtail extension 112, may optionally extend from the distal end of inflow cage 114. In this regard, when left ventricle LV contracts during systole, the left ventricular wall may contact pigtail extension 112, instead of inflow cage 114, thereby preventing anatomical damage to the ventricular wall. In other instances, the system 100 may not include pigtail extension 112.

When the pump is operated, blood will be pumped in the proximal direction from blood inflow cage 114, through cannula 116, to blood outflow cage 118. In this respect, the intracardiac pump illustrated in FIG. 2 is designed for left heart support. The intracardiac pump, however, may alternatively be configured to pump blood in the leading direction (e.g., for applications where the pump is used for right heart support), in which case cage 118 would operate as the blood inflow cage, and cage 114 would operate as the blood outflow cage.

Intracardiac pump and repositioning sheath assembly 110 is typically introduced into the vasculature of a patient via an introducer sheath assembly 150. As depicted in FIG. 2, introducer sheath assembly 150 may include an introducer sheath body 152, a hub 154, and an irrigation line 156. Introducer sheath body 152 may be any suitable type of sheath, for example, a tear-away sheath configured to be ripped or peeled along its length and discarded after the repositioning sheath 126 has been inserted into the vasculature of a patient. Alternatively, introducer sheath body 152 may be an expandable sheath configured to expand radially to allow the passage of the largest portions of the intracardiac pump, and to then contract thereafter, in which case the introducer sheath may remain within the patient even after the intracardiac pump has been introduced into the patient.

Once repositioning sheath 126 has been fully inserted into the patient, the clinician may secure the sheath to the patient at or near the incision (e.g., adjacent the femoral artery) using butterfly 130. In this regard, butterfly 130 may be affixed to the patient, using adhesives or sutures, to secure the repositioning sheath 126 and securement device 132 relative to the patient. With repositioning sheath 126 secured to the patient, the clinician may then advance the intracardiac pump to the target site and use securement device 132 to restrict further movement of the intracardiac pump after it has been properly positioned within heart H.

As previously mentioned, securement device 132 may be a conventional Tuohy-Borst type device, although any other securement device known in the art may be used. Conventional Tuohy-Borst type devices include a barrel that may be rotated in a first direction (e.g., a clockwise direction) to clamp the securement device about catheter 122, which in turn restricts movement of the catheter. In order to extend or retract catheter 122, the clinician must first rotate the Tuohy-Borst type device in a second direction (e.g., a counterclockwise direction) to reduce the clamping force imposed by securement device 132. Rotating Tuohy-Borst type devices can be tedious. This challenge is exacerbated by the fact that butterfly 130 is secured to the skin of the patient, which in turn results in securement device 132 being in close contact with the skin of the patient. Accordingly, it can be difficult to access and rotate the barrel of the Tuohy-Borst type device, which impedes the clinician's ability to easily transition the Tuohy-Borst type device between an unlocked position, in which catheter 122 is freely slidable, and a locked position in which the catheter is stabilized.

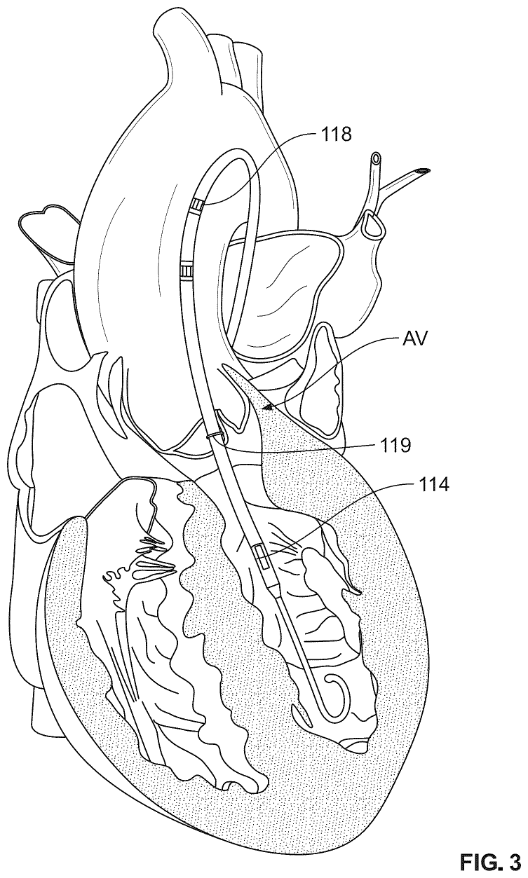

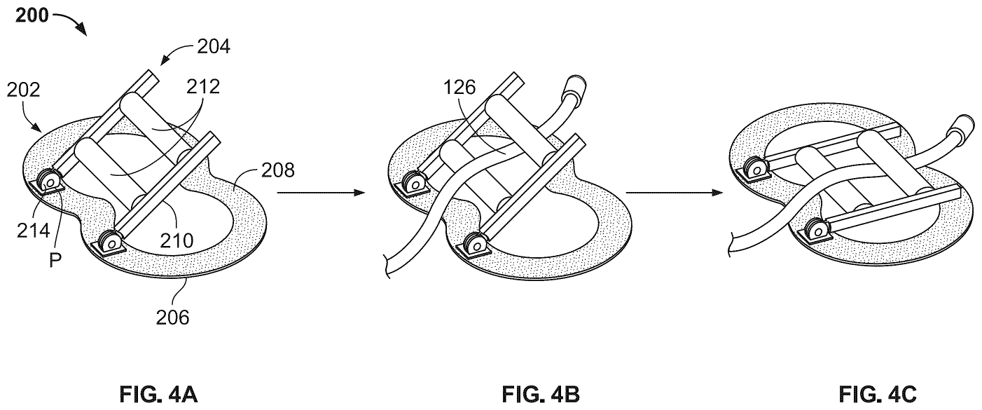

FIGS. 4A-4C illustrate a securement device 132 in the form of catheter fixation device 200 configured to efficiently transition between unlocked and locked positions. Catheter fixation device 200 may be incorporated into intracardiac pump and repositioning sheath assembly 110. More specifically, catheter fixation device 200 may replace the conventional Tuohy-Borst type device, or both the conventional Tuohy-Borst type device and butterfly 130. In other embodiments, catheter fixation device 200 may be used in conjunction with the system 100. For example, catheter fixation device 200 may be positioned proximal to the securement device 132 of intracardiac pump and repositioning sheath assembly 110 as depicted in FIG. 2.

It will be appreciated that catheter fixation device 200 may be used with any intracardiac blood pump or other medical device secured to a delivery system such as a repositioning catheter for which a clinician wishes to selectively restrict movement. Examples of such systems include: angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, or any other venous or arterial based introduced catheters and devices.

Catheter fixation device 200 may include a base 202 and a ladder-lock 204. Base 202 may include a skin contacting surface 206 configured to be secured to the skin of the patient, and an upper surface 208 opposite to the skin contacting surface. In one example, base 202 may be an adhesive pad and skin contacting surface 206 may be coated with a medical grade adhesive to securely fix the base to skin. Alternatively, base 202 may be formed of a fabric, a rubber, or a plastic material defining one or more eyelets sized and arranged to receive a suture, thereby allowing the base to be sutured to skin. Thus, base 202 performs the same function as butterfly 130 in that the base securely fastens the proximal end of repositioning sheath 126 to the skin of the patient, adjacent the incision, through which the catheter 122 is introduced.

Ladder-lock 204 may be movable relative to base 202 between an unlocked position, as shown in FIG. 4B, and a locked position, as shown in FIG. 4C. Ladder-lock 204 may include a set of rails 210 and one or more rungs 212 extending between the rails. Although FIGS. 4A-4C illustrate ladder-lock 204 as including two rungs 212, the ladder-lock may include any number of rungs including a single rung, or three or more rungs. When ladder-lock 204 includes a plurality of rungs 212, the rungs are spaced apart from one another at an adequate distance to allow repositioning sheath 126 to be weaved therebetween. For example, when ladder-lock 204 includes two or more rungs 212, as shown in FIGS. 4A-4C, repositioning sheath 126 may be disposed over a first one of the rungs adjacent feet 214, and underneath a second one of the rungs further away from the feet. Nevertheless, repositioning sheath 126 may be weaved over and underneath the rungs 212 in a multitude of different patterns so long as repositioning sheath is clamped between at least one of the rungs and base 202 and/or skin of the patient when ladder-lock is in the locked position. In one embodiment, one or more of the rungs 212 may each have a gap formed between a respective free end of the rung and the adjacent rail 210. In such embodiments, a physician may pass the repositioning sheath 126 and/or catheter of the device through the one or more gaps to assist with the weaving of repositioning sheath 126 through two or more rungs 212 of ladder lock 204. For example, one or more gaps may allow for a catheter having a larger and/or fixed distal end, which may provide difficultly for the physician to weave through one or more of the rungs 212, be positioned within ladder locker 204 through the one or more gaps. Additionally, as will be appreciated, rails 210 and rungs 212 may be made of any suitable material. In some embodiments, rails 210 and rungs 212 may be made of the same material. In other embodiments, rails 210 and rungs 212 may be made of different materials. For example, rails 210 may be made of a first material and rungs 212 may be made of a second material more flexible than the first material.

FIGS. 4A-4C illustrate rails 210 extending continuously from respective feet 214. In one embodiment, one or more of the rails 210 may have one or more discontinuities. In such embodiments, a physician may pass the repositioning sheath 126 and/or catheter of the device through the one or more discontinuities to position the catheter fixation device 200 in a desired location. In some embodiments, the one or more discontinuities may be on the same rail 210. In other embodiments, each rail 210 may have the same or a different number of discontinuities.

Feet 214 that may be pivotably connected to base 202 about a pivot point P to transition the ladder-lock between the unlocked and locked positions. A tension bearing mechanism may be provided in rails 210, for example, in feet 214. The tension bearing mechanism may be a resilient material such as a spring, or other structure capable of performing the same function. Consequently, a user is required to apply an increased force (e.g., torque) to transition ladder-lock 204 between the unlocked and locked positions. In this regard, ladder-lock 204 will remain in a steady state unless intentionally moved by a user such that when the ladder-lock is in the locked position, the ladder-lock can impart a sufficient clamping force upon repositioning sheath 126 to prevent the catheter 122 from extending or retracting within the cardiovascular system of the patient.

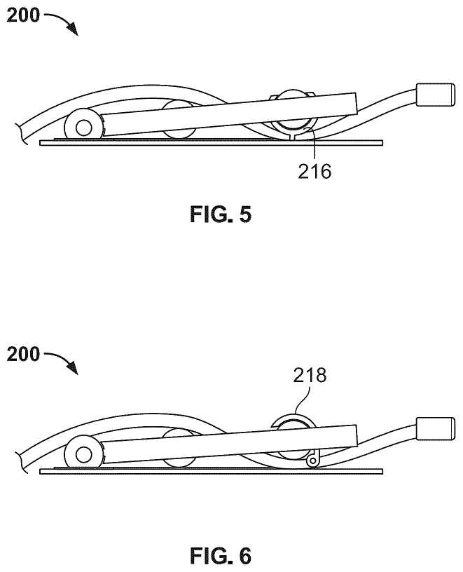

Turning now to FIGS. 5 and 6, catheter fixation device 200 may optionally include a fastener 216 or a latch 218 in replace of the tensioning mechanism or in addition to the tensioning mechanism to assist in retaining the ladder-lock 204 in the locked position. With specific reference to FIG. 5, fastener 216 may be generally U-shaped and be formed of a resilient material sized and arranged to receive a rung 212. Consequently, as ladder-lock 204 is transitioned to the locked position, the rung 212 will force the U-shaped fastener 216 to expand to receive the rung. Once rung 212 is positioned within fastener 216, the resilient fastener may return to its natural state, thereby mechanically securing the rung and, in turn, ladder-lock 204 in the closed position. In the locked position, rung 212 of ladder-lock 204 compresses repositioning sheath 126, which in turn, imparts a clamping force on catheter 22, thereby preventing the catheter from extending or retracting within the cardiovascular system of the patient. The rung 212 may be removed from fastener 216 upon sufficient force applied by a user.

With reference to FIG. 6, latch 218 may be movably connected to base 202 to cover rung 212. For example, latch 218 may be pivotable or slidable relative to base 202. In this regard, after ladder-lock 204 has been transitioned to the locked position, latch 218 may be moved over the top of rung 212 to prevent the ladder-lock from reverting back to the unlocked position. Again, it will be appreciated that when ladder-lock 204 is in the locked position, rung 212 compresses repositioning sheath 126, which imparts a clamping force on catheter 122, and prevents the catheter from extending or retracting within the cardiovascular system of the patient. To return ladder-lock 204 to the unlocked position, a user must first unlatch latch 216, before pivoting the ladder-lock away from base 202.

In use, catheter fixation device 200 may be used in conjunction with an intracardiac pump and repositioning sheath to efficiently reposition the sheath and, in turn, the intracardiac pump. Although use of catheter fixation device 200 is described hereinafter in connection with a percutaneous intracardiac pump that is delivered to the left-side of the heart using a transfemoral approach, it will be appreciated that the catheter fixation device 200 may be used in conjunction with percutaneous intracardiac devices providing right heart support, as well as other medical devices secured to a delivery system (e.g., a repositioning catheter) for which a clinician wishes to selectively restrict movement.

Initially, it is noted that the physician may use more than one catheter fixation device 200 to restrict catheter movement. For example, in some embodiments, the physician may position a first catheter fixation device directly proximal a second catheter fixation device. In other embodiments, the physician may position a first catheter fixation device a distance away from a second catheter fixation device. In such embodiments, multiple points of fixation may allow portions of the catheter to remain fixed while allowing specific portions of the catheter to move. For example, the one or more catheter fixation devices may be selectively positioned to allow the patient to ambulate while not displacing the intracardiac pump positioning.

First, a physician may make an incision into skin to provide an access point to the femoral artery of the patient. The distal end of introducer sheath assembly 150 may then be inserted through the incision and into the femoral artery. Next, with the intracardiac pump positioned within repositioning sheath 126 of modified intracardiac pump and repositioning sheath assembly 110, the clinician may weave the repositioning sheath over and underneath rungs 212 of ladder-lock 204 when ladder-lock 204 is in the unlocked position. For example, repositioning sheath 126 may be weaved over the first rung disposed adjacent to the feet 214 of ladder-lock 204 and underneath a second rung disposed further from the feet. The clinician may then insert the distal end of repositioning sheath 126 through the hub 154 of introducer sheath assembly 150 and advance the repositioning sheath through introducer sheath body 152. In this regard, introducer sheath body 152 will act as the conduit for repositioning sheath 126 to enter the vasculature of the patient.

With repositioning sheath 126 fully advanced through introducer sheath body 152, the clinician may then secure the base 202 of catheter fixation device 200 to the skin of the patient adjacent the incision. When introducer sheath assembly 150 is formed as a tear-away sheath, the introducer sheath assembly may then be removed by breaking hub 154 and tearing introducer sheath body 152 along its length after repositioning sheath 126 has been inserted into the vasculature of the patient. On the other hand, if introducer sheath assembly 150 is expandable, it may remain in the body, either surrounding some or all of the repositioning sheath 126.

Under echocardiography and/or other traditional imaging techniques, the clinician may operate handle 138 to advance catheter 122 toward the target site. The clinician may then continue to track the intracardiac pump through the vasculature of the patient and into heart H via aortic arch AA. Using fluoroscopy, the physician may align the marking 119 on cannula 116 within the aortic valve AV as shown in FIG. 3. In this position, inflow cage 114 may be centrally positioned within left ventricle LV and outflow cage 118 may be sufficiently positioned within aorta A.

After the clinician has confirmed that the intracardiac pump is properly positioned within the heart H of the patient, ladder-lock 204 may be transitioned from the unlocked position to the locked position to clamp repositioning sheath 126 about catheter 122, thereby preventing the catheter from moving with respect to the cardiovascular system of the patient. Thereafter, the intracardiac pump may be turned on to suction blood from the left ventricle LV and into inflow cage 114, through cannula 116, and out from outflow cage 118 into aorta A.

In some embodiments, the physician may alternatively insert and position the intracardiac pump as described herein and then pass the repositioning sheath 126 and/or the catheter through the one or more discontinuities of the fixation device 200. Once the fixation device 200 is in the desired location on the length of the repositioning sheath 126 and/or the catheter, the clinician may then secure the base 202 to the skin of the patient. With base 202 secured to the skin, the ladder-lock 204 may be transitioned from the unlocked position to the locked position. In the event the clinician deems it appropriate to secure additional locations along the length of the repositioning sheath 126 and/or the catheter, the clinician may add additional catheter fixation devices 200 by passing the repositioning sheath 126 and/or catheter through the discontinuities without needing to stop support and/or remove the intracardiac pump.

In some embodiments, more than one catheter-based device may pass through the introducer sheath assembly 150. In such embodiments, each catheter of the respective catheter-based device may pass through catheter fixation device 200. In other embodiments, each of the one or more catheter-based devices may pass through one or more sheath assemblies. In such embodiments, one or more catheter fixation devices 200 may be used. For example, a first catheter may pass through a first sheath assembly and through a first catheter fixation device and a second catheter may pass through a second sheath assembly and through a second catheter fixation device. In some embodiments, the one or more sheath assemblies may include one or more introducer sheath assemblies 150. As will be appreciated, any suitable number of sheath assemblies and/or catheter fixation devices may be used to accommodate one or more catheter-based devices through one or more access sites.

As mentioned herein, however, operation of the pump and/or movement of the patient may cause catheter 122 and the intracardiac pump to move, or migrate, from the intended position due, in part, to a change in slack of the catheter within the cardiovascular system of the patient. For this reason, the clinician may monitor the position of the intracardiac pump throughout its use. In the event that the clinician deems it appropriate to reposition the intracardiac device within the heart H of the patient, the clinician may apply a force that overcomes the tensioning mechanism to pivot the ladder-lock 204 away from base 202 and to the unlocked position. In the unlocked position, the clinician may operate handle 138 to reposition catheter 122 as desired, before again transitioning ladder-lock 204 to the locked position.

It will be appreciated that ladder-lock 204 is easily accessible as it is positioned above the upper surface 208 of base 202 and that it may be transitioned between unlocked and locked positions in a single motion. As a result, ladder-lock 204 simplifies locking and unlocking a repositioning catheter when desired compared to securement devices including conventional Tuohy-Borst type devices.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for securing a catheter carrying a medical device, comprising:
a base defining a skin contacting surface; and
a ladder-lock coupled to the base, the ladder-lock including a pair of rails and one or more rungs extending between the rails, each of the rails being pivotably attached to the base such that the ladder-lock is pivotable from an unlocked position in which the catheter is slidable within a cardiovascular system of a patient, and a locked position in which the catheter is fixed relative to the cardiovascular system.

2. The device of claim 1, wherein the base is an adhesive pad, and the skin contacting surface of the adhesive pad comprises a medical grade adhesive configured to be adhered to skin of the patient.

3. The device of claim 1, wherein the base comprises a material configured to be sutured to skin of the patient.

4. The device of claim 1, wherein the material comprises a fabric, a rubber, or a medical grade plastic defining one or more eyelets.

5. The device of claim 1, wherein the one or more rungs include a plurality of rungs, and the plurality of rungs are spaced apart from one another such that the catheter is configured to extend over at least a first one of the rungs and underneath at least a second one of the rungs.

6. The device of claim 1, further comprising a spring biasedly arranged against one of the rails to stabilize the ladder-lock against unintended movement.

7. The device of claim 1, further comprising at least one fastener to secure the ladder-lock in the locked position.

8. The device of claim 1, wherein the fastener is substantially U-shaped and formed of a resilient material, the fastener being arranged to receive the one or more rungs to mechanically secure the ladder-lock in the locked position.

9. The device of claim 1, further comprising at least one latch movably arranged to cover at least one of the rails or the one or more rungs to secure the ladder-lock in the locked position.

10. An assembly, comprising:
a catheter for inserting an intravenous medical device into a cardiovascular system of a patient; and
an apparatus for stabilizing the catheter within the cardiovascular system of the patient, comprising:
a base defining a skin contacting surface; and
a ladder-lock coupled to the base, the ladder-lock including a pair of rails and one or more rungs extending between the rails, each of the rails being movably attached to the base and movable between a first position in which the catheter is slidable within the cardiovascular system and a second position which the catheter is compressed by the ladder-lock and fixed relative to the cardiovascular system.

11. The assembly of claim 10, wherein the catheter is a repositioning catheter.

12. The assembly of claim 10, wherein the intravenous medical device is an intracardiac pump.

13. The assembly of claim 10, wherein the base is an adhesive pad.

14. The assembly of claim 10, wherein the base comprises a material configured to be sutured to skin of the patient.

15. The assembly of claim 10, wherein the one or more rungs includes a plurality of rungs, and the one or more rungs are spaced apart from one another such that the catheter is configured to be weaved over at least a first one of the rungs and underneath at least a second one of the rungs.

16. A method of delivering and stabilizing a medical device within a cardiovascular system of a patient, the method comprising:
inserting a catheter holding the medical device through the cardiovascular system of the patient;
positioning the medical device at a target site;
securing a base of a catheter fixation apparatus to skin of the patient; and
transitioning a ladder-lock attached to the base from an unlocked position in which the catheter is slidable within the cardiovascular system of the patient to a locked position in which the catheter is compressed by the ladder-lock to fix the catheter relative to the cardiovascular system of the patient.

17. The method of claim 16, wherein the medical device is an intracardiac pump and the target site is across a native heart valve.

18. The method of claim 16, further comprising:
determining that the intracardiac pump is mispositioned;
transitioning the ladder-lock from the locked position to the unlocked position;
sliding the catheter to reposition the intracardiac pump within the native valve; and
transitioning the ladder-lock from the unlocked position to the locked position.

19. The method of claim 16, further comprising securing the base to skin of the patient via an adhesive or a suture.

20. The method of claim 16, further comprising weaving the catheter over a first rung of the ladder-lock and underneath a second rung of the ladder-lock.
